(19) United States
(12) Patent Application Publication
(10) Pub. No.: US 2020/0096503 A1
(43) Pub. Date: Mar. 26, 2020

(54) CELL MEMBRANE COATED MAGNETIC NANOPARTICLES AND ASSAYS FOR IDENTIFICATION OF TRANSMEMBRANE PROTEIN-BINDING COMPOUNDS

(71) Applicant: The Board of Trustees of The University of Alabama, Tuscaloosa, AL (US)

(72) Inventors: Lukasz Ciesla, Northport, AL (US); Yuping Bao, Tuscaloosa, AL (US)

(21) Appl. No.: 16/523,454

(22) Filed: Jul. 26, 2019

Related U.S. Application Data
(60) Provisional application No. 62/736,762, filed on Sep. 26, 2018.

(51) Int. Cl. G01N 33/543; A61K 36/00

(52) U.S. Cl. CPC G01N 33/54346; B82Y 5/00; A61K 36/00; G01N 33/54333

(57) ABSTRACT

Disclosed herein are nanostructures comprising a cell membrane-derived material comprising a target membrane protein; and one or more magnetic nanoparticles; wherein the cell membrane-derived material encapsulates the one or more magnetic nanoparticles. Also disclosed are methods of screening a sample for a binding agent, the method comprising contacting a sample comprising a binding agent with a nanostructure to form a mixture, the nanostructure comprising a cell membrane-derived material comprising a target membrane protein; and one or more magnetic nanoparticles; wherein the cell membrane-derived material encapsulates the one or more magnetic nanoparticles; and separating the nanostructure and any binding agent bound thereto from the mixture with a magnet.

### CROSS-REFERENCE TO RELATED APPLICATIONS

This application claims the benefit of and priority to U.S. Provisional Application Ser. No. 62/736,762 filed on Sep. 26, 2018, the disclosure of which is expressly incorporated herein by reference in its entirety.

### STATEMENT REGARDING FEDERALLY SPONSORED RESEARCH

This invention was made with government support under Grant No. DMR-1149931 awarded by the National Science Foundation. The Government has certain rights in the invention.

### FIELD

The disclosure generally relates to nanostructures and assays using nanostructures to discover transmembrane protein-binding compounds such as pharmacologically active drugs, antibiotics for resistant strains, or pesticides from natural resources.

### BACKGROUND

Natural compounds have proven to be a rich source of new biologically active compounds. Numerous plant/bacterial/fungal metabolites evolved in response to different abiotic and biological stresses, for example, herbivore attacks. Noxious phytochemicals warding off herbivores stimulate cellular and metabolic pathways that evolved to protect cells. Many of these pathways are evolutionary conserved and humans share them with simple herbivorous animals. Crude mixtures such as these phytochemicals constitute a practically untapped source of evolutionary-designed compounds that may become another generation of drugs. Technical constraints of currently approved high-throughput screening assays prevent the identification of pharmacologically active compounds from complex matrices. Traditional approaches used in phytochemistry and pharmacology laboratories are time consuming and costly. These approaches usually lead to dereplication, thereby, isolation of the most abundant compounds and require high resource commitment to achieve targeted goals.

Approximately half of current drug discovery programs target transmembrane proteins (ion channels, GPCRs, enzyme-linked receptors, transporters, etc.). Immobilization of fully functional transmembrane proteins on magnetic beads is a very challenging task and previous attempts of using magnetic beads with immobilized receptors have been unsuccessful because of significant nonspecific binding of compounds. What are needed are tools and assays which can identify unknown ligands of transmembrane receptors from a complex matrix (a crude mixture). Such tools and assays would be extremely advantageous if they are adaptable for use with any transmembrane receptor from any living species, and for use to screen numerous compounds from an array of complex mixtures. Tools and assays having these capabilities represent the potential to discover potentially therapeutic compounds effective against numerous diseases.

As an example, Alzheimer's disease (AD) and other neurodegenerative ailments have emerged as great medical challenges of the 21st century. Despite many years of research, the exact causes of AD remain unknown (Lu et al., Nature. 2014; 507(7493):448-54). The lack of understanding of AD has caused an absence of effective pharmacological approaches in the prevention and treatment of Alzheimer's disease. Based on all available data, it can be postulated that known hallmarks of neurodegeneration, including senile plaques are not solely responsible for development and progression of AD. Id. Very recently, evolutionary origins of aging-related diseases were suggested (Chen et al., Cell Syst. 2018; 6(5):604-11 e4). Neuron-specific enhancers have been identified as benefiting brain development, but at the same time increasing human brain susceptibility for neurodegenerative diseases. Interestingly, one of these enhancers was found to promote gene expression suppressed by re-1 silencing transcription factor (REST), which was shown previously to be neuroprotective (Lu et al., Nature. 2014; 507(7493):448-54).

New data further accentuate the importance of studying adaptive and conserved cellular signaling and metabolic pathways that have evolved to protect cells (including neurons) and organs from different forms of biological stress (Lu et al., Nature. 2014; 507(7493):448-54; Chen et al., Cell Syst. 2018; 6(5):604-11 e4; Mattson et al., Nat Rev Neurosci. 2018; 19(2):63-80; Lee et al., Pharmacol Rev. 2014; 66(3):815-68). These pathways are part of the stress response system that humans developed and retained in response to several intermittent environmental challenges, such as: food scarcity, intensive endurance aerobic physical activity, and noxious phytochemicals (Lu et al., Nature. 2014; 507(7493):448-54; Mattson et al., Nat Rev Neurosci. 2018; 19(2):63-80; Lee et al., Pharmacol Rev. 2014; 66(3):815-68; Mattson M P. Sci Am. 2015; 313(1):40-5; Murugaiyah et al., Neurochem Int. 2015; 89:271-80; Mattson M P., Dose-Response. 2014; 12(4):600-18; Mattson et al., Dose Response. 2007; 5(3):174-86; Mattson et al., Neurohormetic phytochemicals: Low-dose toxins that induce adaptive neuronal stress responses. Trends Neurosci. 2006; 29(11):632-9). Some of the identified evolutionary conserved adaptive stress cellular signaling pathways include: Nuclear Factor Erythroid 2-Related Factor 2 Activation pathway (Nrf-2), NF-κB pathway, BDNF signaling pathway, insulin signaling pathway and canonical WNT-β-catenin pathway (Lee et al., Pharmacol Rev. 2014; 66(3):815-68; Nusse et al., Cell. 2017; 169(6):985-99). Stimulation of adaptive cellular and metabolic signaling pathways can result in increased cell resilience and neuroprotection (Mattson et al., Nat Rev Neurosci. 2018; 19(2): 63-80).

Intermittent fasting and intensive endurance aerobic exercise, two of the environmental challenges, have been well studied and their beneficial and neuroprotective effects have been proven (Mattson et al., Nat Rev Neurosci. 2018; 19(2):63-80). Fasting and vigorous exercise have been found to provide neuroprotection by generating ketone bodies, which upregulate the expression of BDNF. Numerous noxious phytochemicals ingested with foodstuffs constitute another group of environmental challenges (Mattson M P., Sci Am. 2015; 313(1):40-5; Mattson M P., Dose-Response. 2014; 12(4):600-18). Many secondary plant metabolites evolved in response to different abiotic and biological stresses, for example herbivore attacks. Noxious phytochemicals warding off herbivores stimulate cellular and metabolic pathways that evolved to protect cells. Many of these pathways are evolutionary conserved and humans share them with simple herbivorous animals.

### SUMMARY

The disclosed subject matter relates to nanostructures, methods to make nanostructures, and methods of screening a sample for a binding agent (e.g., a pharmacologically active agent) using nanostructures.

In another aspect, provided herein are methods of screening a sample for a binding agent, the method comprising contacting a sample comprising a binding agent with a nanostructure to form a mixture, the nanostructure comprising a cell membrane-derived material comprising a target membrane protein; and one or more magnetic nanoparticles; wherein the cell membrane-derived material encapsulates the one or more magnetic nanoparticles; and separating the nanostructure and any binding agent bound thereto from the mixture with a magnet.

In some embodiments, the sample comprises a biological extract, or a crude mixture of unknown components. In some embodiments, the cell membranes can be prepared from human cells, animal cells, insect cells, or bacterial cells. In some embodiments, the biological extract is from a plant. In some embodiments, the sample comprises a smoke condensate. In some embodiments, the method further comprises separating the binding agent from the target membrane protein, which can be performed by combining a solvent (e.g., an organic solvent) in an amount up to about 10 percent with the nanostructure. In some embodiments, the method can further comprise identifying the binding agent bound to the target membrane protein. In some embodiments, the binding agent is a phytochemical. In some embodiments, the method can further comprise determining a pharmacological activity of the binding agent in a cell-based assay. In some embodiments, the binding agent activates a BDNF or a Wnt-β-catenin pathway. In some embodiments, the binding agent is an antibiotic (e.g., an antibiotic which disrupts a biofilm). In some embodiments, the binding agent is a pesticide (e.g., an environmentally-friendly pesticide). In some embodiments, the method can further comprise repeating each step reusing the same nanostructure. In some embodiments, the method comprises substantially no non-specific binding between the sample and the magnetic nanoparticles. In some embodiments, the method identifies a pharmacological agent useful for treating a disease, such as a neurodegenerative disease. In some embodiments, the method can further comprise administering the binding agent to a subject with a disease.

In one aspect, disclosed herein are nanostructures comprising a cell membrane-derived material comprising a target membrane protein; and one or more magnetic nanoparticles; wherein the cell membrane-derived material encapsulates the one or more magnetic nanoparticles. In some embodiments, the nanostructure comprises the cell membrane-derived material and the magnetic nanoparticles in a weight ratio ranging from about 1:100 to about 1:600. In some embodiments, the nanostructure comprises from about 200 to about 1,000 magnetic nanoparticles, and/or comprises a diameter of from about 100 nm to about 1,000 nm. In some embodiments, the target membrane protein comprises a transmembrane protein, which can comprise TrkB or FZD1, a nicotine receptor, an ectopic olfactory receptor, or a TRP channel protein. In some embodiments, the target membrane protein comprises TrkB or FZD1. In some embodiments, the cell membrane-derived material is obtained from a cell membrane of a biological cell. In some embodiments, the cell membranes are from a human cell, an animal cell, an insect cell, or a bacterial cell. In some embodiments, a human cell, for example a neuronal cell, is selected for preparation of the cell membrane-derived material. In some embodiments, the biological cell comprises an artificial vector encoding the target membrane protein. In some embodiments, the magnetic nanoparticles comprise a surface coating that is negatively charged, which can comprise a moiety selected from tannic acid, a gluconic acid, a citric acid, a glutathione, a quinic acid, a lactobionic acid, a dopamine, and a polyacrylic acid.

In another aspect, provided herein are methods to make a nanostructure comprising contacting a cell membrane-derived material with one or more magnetic nanoparticles to form a mixture, wherein the nanostructure comprises a cell membrane-derived material comprising a target membrane protein; and one or more magnetic nanoparticles; wherein the cell membrane-derived material encapsulates the one or more magnetic nanoparticles.

Additional aspects and advantages of the disclosure will be set forth, in part, in the detailed description and any claims which follow, and in part will be derived from the detailed description or can be learned by practice of the various aspects of the disclosure. The advantages described below will be realized and attained by means of the elements and combinations particularly pointed out in the appended claims. It is to be understood that both the foregoing general description and the following detailed description are exemplary and explanatory only and are not restrictive of the disclosure.

### BRIEF DESCRIPTION OF THE DRAWINGS

The accompanying drawings, which are incorporated in and constitute a part of this specification, illustrate certain examples of the present disclosure and together with the description, serve to explain, without limitation, the principles of the disclosure. Like numbers represent the same element(s) throughout the figures.

FIG. 1 is a schematic depicting an overview of cell-membrane coated nanostructures (CMNP) formation and compound screening process: (1) cells expressing targeted proteins, (2) iron oxide nanoparticles, (3) cell membrane fragments obtained after cell lysis, (4) cell membrane encapsulated nanoparticles-CMNPs, and (5) screening process with CMNPs.

FIG. 2 is a typical TEM image of CMNPs. CMNPs are labeled as typical CMNPs (“CMNP”), non-spherical CMNP, or small-sized CMNP.

FIG. 3A and FIG. 3B are schematics depicting an overview of the ligand fishing process. FIG. 3A shows the detailed ligand fishing procedures. FIG. 3B shows an example of results from HPLC analysis.

FIG. 4 is a graph showing an example HPLC analysis of fishing results using CMNPs with nicotinic receptors and an artificial mixture of known binders and non-binders.

FIG. 5A and FIG. 5B are graphs showing an example HPLC analysis of fishing results using CMNPs with nicotinic receptors and tobacco smoke condensate.

FIGS. 6A and 6B are TEM images of CMNPs comprising TrkB receptors (FIG. 6A) and a closer view of the CMNP comprising the TrkB receptors (FIG. 6B), where the cell membrane shell is clearly seen in FIG. 6B.

FIG. 7A is a graph showing HPLC-ESI-MS chromatograms (negative ionization mode) of fishing experiments using CMNPs with TrkB receptors and artificial mixture: washing and elution profiles showing the binding patterns of binder and non-binders.

FIG. 7B is a graph showing HPLC-ESI-MS chromatograms (positive ionization mode) of fishing experiments using CMNPs with TrkB receptors and artificial mixture: washing and elution profiles showing the binding patterns of nicotine.

FIG. 8 is a graph showing HPLC-ESI-MS chromatograms (negative ionization mode; ATP m/z 505.8; ADP m/z 425.9) presenting the levels of ATP and ADP after 30 and 60 mins of incubation of CMNPs with 5 mM ATP with known TrkB activator 7,8-dihydroxyflavone (100 μM).

FIG. 9 is a graph showing HPLC-ESI-MS chromatograms (negative ionization mode; ATP m/z 505.8;) presenting the levels of ATP after 30 mins of incubation of CMNPs with 5 mM ATP with and without known TrkB activator 7,8-dihydroxyflavone (100 μM).

FIG. 10 is a graph showing HPLC-ESI-MS chromatograms (negative ionization mode; ATP m/z 505.8; ADP m/z 425.9) presenting the levels of ATP and after 24 hrs of incubation of CMNPs with 5 mM ATP with known TrkB activator 7,8-dihydroxyflavone (100 μM) at 4° C.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

### Terminology

Unless defined otherwise, all technical and scientific terms used herein have the same meaning as commonly understood to one of ordinary skill in the art to which this invention belongs. The following definitions are provided for the full understanding of terms used in this specification.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular nanostructure is disclosed and discussed and a number of modifications that can be made to the nanostructure are discussed, specifically contemplated is each and every combination and permutation of the nanostructure and the modifications that are possible unless specifically indicated to the contrary. Thus, if a class of nanostructures A, B, and C are disclosed as well as a class of nanostructures D, E, and F and an example of a combination nanostructure, or, for example, a combination nanostructure comprising A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

It is understood that the compositions disclosed herein have certain functions. Disclosed herein are certain structural requirements for performing the disclosed functions, and it is understood that there are a variety of structures which can perform the same function which are related to the disclosed structures, and that these structures will ultimately achieve the same result.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

As used in the specification and claims, the singular form “a,” “an,” and “the” include plural references unless the context clearly dictates otherwise. For example, the term “an agent” includes a plurality of agents, including mixtures thereof.

As used herein, the terms “can,” “may,” “optionally,” “can optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

A “control” is an alternative subject, sample, or set of values used in an experiment for comparison purposes. A control can be “positive” or “negative.” A control can also be a collection of values used as a standard applied to one or more subjects (e.g., a general number or average that is known and not identified in the method using a sample).

“Peptide,” “protein,” and “polypeptide” are used interchangeably to refer to a natural or synthetic molecule comprising two or more amino acids linked by the carboxyl group of one amino acid to the alpha amino group of another. The amino acids may be natural or synthetic, and can contain chemical modifications such as disulfide bridges, substitution of radioisotopes, phosphorylation, substrate chelation (e.g., chelation of iron or copper atoms), glycosylation, acetylation, formylation, amidation, biotinylation, and a wide range of other modifications. A polypeptide may be attached to other molecules, for instance molecules required for function. Examples of molecules which may be attached to a polypeptide include, without limitation, cofactors, polynucleotides, lipids, metal ions, phosphate, etc. Non-limiting examples of polypeptides include peptide fragments, denatured/unstructured polypeptides, polypeptides having quaternary or aggregated structures, etc. There is expressly no requirement that a polypeptide must contain an intended function; a polypeptide can be functional, non-functional, function for unexpected/unintended purposes, or have unknown function. A polypeptide is comprised of approximately twenty, standard naturally occurring amino acids, although natural and synthetic amino acids which are not members of the standard twenty amino acids may also be used. The standard twenty amino acids include alanine (Ala, A), arginine (Arg, R), asparagine (Asn, N), aspartic acid (Asp, D), cysteine (Cys, C), glutamine (Gln, Q), glutamic acid (Glu, E), glycine (Gly, G), histidine, (His, H), isoleucine (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y), and valine (Val, V). The terms “polypeptide sequence” or “amino acid sequence” are an alphabetical representation of a polypeptide molecule.

“Pharmaceutically acceptable” component can refer to a component that is not biologically or otherwise undesirable, e.g., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., Alzheimer's disease).

“Subject” includes animals such as mammals, including, but not limited to, primates (e.g., humans), cows, sheep, goats, horses, dogs, cats, rabbits, rats, mice and the like. In some embodiments, the subject is a human.

“Therapeutically effective amount” of a composition (e.g. a composition comprising an agent) refers to an amount that is effective to achieve a desired therapeutic result. In some embodiments, a desired therapeutic result comprises any amelioration of the symptoms of a neurodegenerative disease. In some embodiments, a desired therapeutic result comprises activation of a Nuclear Factor Erythroid 2-Related Factor 2 Activation pathway (Nrf-2), a NF-κB pathway, a Wnt-β-catenin pathway, a BDNF pathway, or any combination thereof. In some embodiments, a desired therapeutic result comprises increased survival of neuronal cells compared to a control. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

“Vector” refers to a DNA construct containing a DNA expression cassette (e.g., a gene) which is operably linked to one or more expression control sequences capable of effecting the expression of the DNA expression cassette within the DNA construct in a suitable cell or host. Expression control sequences include, but are not limited to, a promoter to effect transcription, an optional operator sequence to control or modify such transcription, a sequence encoding suitable mRNA ribosome binding sites, and sequences which control the termination of transcription and translation. The vector may be a plasmid, a phage nanoparticle, or simply a potential genomic insert. Once transformed into a suitable host, the vector may replicate and function independently of the host genome, or may in some instances, integrate into the genome itself. A plasmid is the most commonly used form of a vector; however, the invention is intended to include such other forms of vectors which serve equivalent function as and which are, or become, known in the art.

### Nanostructures

It is understood that the nanostructures of the present disclosure can be used in combination with the various compositions, methods, products, and applications disclosed herein.

The variety and complexity of samples (e.g., biological extracts) having numerous unknown components in unknown amounts makes it difficult to identify component(s) within the sample which may have pharmacological activity. Current approaches do not allow for direct identification of such active compounds in complex mixtures. The disclosure herein addresses needs in the art by providing for nanostructures and assays using nanostructures which allow for the identification of components in a complex mixture which bind to membrane receptors. Such components may activate or inhibit one or more cellular responses by binding the membrane receptor and thus, can be pharmacologically active agents. The assay can be performed using very crude samples such as unpurified cellular, tissue, or biological extracts. The nanostructures are also advantageous because they can be separated from crude samples after receptor binding, exhibit little to no non-specific binding which generally plagues magnetic nanoparticle-based assays, and can be reused in iterations of the disclosed screening assays.

Disclosed herein are nanostructures comprising a cell membrane-derived material comprising a target membrane protein; and one or more magnetic nanoparticles; wherein the cell membrane-derived material encapsulates the one or more magnetic nanoparticles. In some instances, the nanostructures are referred to herein as cell-membrane coated nanoparticles (CMNPs).

In some embodiments, the cell membrane-derived material fully encapsulates the magnetic nanoparticles. Encapsulation of the magnetic nanoparticles can shield the magnetic nanoparticles from components in the sample. Encapsulation can reduce or essentially eliminate non-specific binding of sample components to the magnetic nanoparticles. Thus, encapsulation can be similar to that of a liposome which encapsulates and shields intraliposomal aqueous components.

By cell membrane-derived material, it is meant that the material comprises components of a cell membrane. For example, the material comprises cell membrane lipids, proteins (e.g., transmembrane receptors, membrane transporters, and lipid-anchored proteins), sterols, glycolipids, glycoproteins, and other components which are integrated within or bound to a cell membrane. The cell membrane-derived material can further contain additional non-cell components such as detergents used to extract cell membrane components, proteases inhibitors to protect integrity of membrane proteins, and buffer components (e.g., salts, glycerol, buffering agents such as Tris-HCl, HEPES, potassium phosphate). In some embodiments, the cell membrane-derived material is removed of substantially all cytosolic components of the cell. For example, cellular debris can be removed by centrifugation and subsequent collection of the cell membrane-derived material as a membranous fraction.

As the cell membrane-derived material comprises components of a cell membrane, the material comprises numerous membrane proteins embedded within or attached to the cell membrane (collectively referred to herein as “membrane proteins”). One or more membrane proteins may be of interest for identification of a binding agent (e.g., a pharmacologically active agent). For instance, a membrane protein of a cell can be associated with a cellular response or signaling pathway that a method user wishes to exploit. Binding of an agent to such a target membrane protein may activate or inhibit that cellular response or signaling pathway. The target membrane protein can be any membrane protein. When formed into the nanostructure, at least a portion of the target membrane protein is typically accessible on the surface of a nanostructure (e.g., accessible to a binding agent). However, any given nanostructure can comprise the target membrane protein as a mix of correctly and incorrectly oriented proteins, or alternatively substantially all the target membrane protein can be in the correct orientation. By correct orientation, it is meant the portion of the target membrane protein which is accessible on the surface of the nanostructure is substantially the same portion which is accessible on the surface of a biological cell comprising that protein. By incorrect orientation, it is meant the portion of the target membrane protein which is accessible on the surface of a biological cell is positioned to the interior of the nanostructure and is thus generally inaccessible to a binding agent on the outer surface of the nanostructure.

Generally, the target membrane protein is a native membrane protein of the cell from which the cell membrane-derived material is obtained. However, this need not always be the case, and the target membrane protein can be a heterologous or orthologous protein, or an unrelated foreign protein, present in the cell membrane of the cell from which the cell membrane-derived material is obtained (e.g., the target membrane protein is a protein expressed from a vector, or is inserted into the cell membrane or cell-derived membrane material by hydrophobic interaction). In some embodiments, the cell supplying the cell membrane-derived material comprises a vector (e.g., plasmid or viral vector) encoding the target membrane protein. In some embodiments, the vector is used to overexpress the target membrane protein and thus, the cell can comprise an increased amount of the target membrane protein compared to a control cell. In some embodiments, the control cell is a cell of the same cell type but does not contain the vector encoding the target membrane protein (or contains an empty vector). In some embodiments, the cell supplying the cell membrane-derived material can comprise the target membrane protein in an amount which is at least 2-fold, at least 3-fold, at least 4-fold, at least 5-fold, at least 10-fold, at least 20-fold, at least 50-fold, at least 100-fold, at least 500-fold, or at least 1,000-fold more compared to a control cell.

In some embodiments, the target membrane protein comprises a transmembrane protein or a transmembrane receptor protein. In some embodiments, the target membrane protein is a complex of two or more macromolecules (e.g., a multi-protein transmembrane receptor complex having tertiary or quaternary structure). In some embodiments, the target membrane protein is associated with a biological or cellular pathway. In some embodiments, the target membrane protein activates or inhibits a biological or cellular pathway associated with a neurological disease. In some embodiments, the target membrane protein activates or inhibits a Nuclear Factor Erythroid 2-Related Factor 2 Activation pathway (Nrf-2), a NF-κB pathway, a Wnt-β-catenin pathway, a BDNF pathway, or any combination thereof. In some embodiments, the target membrane protein activates or inhibits a Wnt-β-catenin pathway or a BDNF pathway. In some embodiments, the target membrane protein comprises a TrkB polypeptide or a FZD1 polypeptide.

Generally, the cell membrane-derived material is obtained from the cell membrane of a biological cell, but can also be obtained from an artificial cell, liposome, planar lipid bilayer, or other artificial membranes containing a target membrane protein. The biological cell supplying the cell membrane-derived material is not particularly limited and can be a prokaryotic or eukaryotic cell. For instance, the biological cell can be a bacterial cell, a protist cell, a fungal cell, a plant cell, or an animal cell. In some embodiments, the biological cell is from a plant, for instance a plant which can be used to produce a smoke condensate. In some embodiments, the biological cell is from an insect, for example a fruit fly (Drosophila), termite, spider, ant, or honey bee. In some embodiments, the biological cell is a mammalian cell, such as a human, dog, cow, horse, mouse, rabbit, etc. In some embodiments, the biological cell is from a primate, particularly a human. The biological cell can be from a mammal of any gender, age, race, creed, ethnicity, socio-economic status, or other general classifiers.

The biological cell supplying the cell membrane-derived material can be selected based on the biological cell's association with a particular organ, tissue, cell-type, or biological pathway. For instance, a method user who wishes to screen for agents which may be pharmacologically active in neurons may opt to select a neuronal cell. In this regard, the specific type of biological cell supplying the cell membrane-derived material is not particularly limited, and can be a cell of any organ, tissue, or cell type.

A method user may opt to analyze a single target membrane protein for potential binding agents. However, two or more target membrane proteins can be used. In some embodiments, the nanostructure comprises two or more, three or more, four or more, five or more, or ten or more target membrane proteins.

The amount of cell membrane-derived material and the magnetic nanoparticles can be important for ensuring encapsulation of the magnetic nanoparticles. In some embodiments, the total cell membrane surface area (A) of 10⁷ cells can be calculated according to Formula 1 below:

A=10⁷×2πR², wherein R=1 μm in this example. Formula 1

The calculated total cell membrane surface area can be used, in some embodiments, to calculate the amount of magnetic nanoparticles to be mixed with the cell membrane-derived material. Using nanostructures having a diameter of about 200 nm as an example, the estimated number of nanostructures (denoted as N_{CMNP}) which can be formed can be estimated by Formula 2 below:

$${N}_{CMNP} = \frac{{10}^{7} \times 2 π {R}^{2}}{4 π {r}^{2}} , where r = 100 nm . \qquad Formula 2$$

The number of nanostructures in this example is thus calculated to be about 10⁹-10¹⁰ depending on membrane recovery. In some embodiments, the minimal ratio of cell membrane-derived material to magnetic nanoparticles can be estimated by dividing the nanostructure volume by the magnetic nanoparticle volume according to Formula 3 below:

$$Ratio = \frac{{r}_{CMNP}^{3}}{{r}_{NP}^{3}} \qquad Formula 3$$

In some embodiments, the nanostructure comprises the cell membrane-derived material and the magnetic nanoparticles in a weight ratio ranging from about 1:50 to about 1:1,000. In some embodiments, the weight ratio between the cell membrane-derived material and the magnetic nanoparticles can range from about 1:75 to about 1:800, from about 1:100 to about 1:600, from about 1:150 to about 1:500, or from about 1:200 to about 1:400. The much higher amounts by weight of magnetic nanoparticles as compared to the amounts by weight of cell membrane-derived material is generally due to the greater density and much heavier weight of magnetic nanoparticles compared to membranous material.

Another important parameter for forming the disclosed nanostructures includes the total number of magnetic nanoparticles encapsulated per nanostructure. Generally, nanostructures having a larger diameter can encapsulate a greater number of magnetic nanoparticles, whereas nanostructures having a smaller diameter can encapsulate a smaller number of magnetic nanoparticles. In some embodiments, a nanostructure can comprise from about 50 to about 5,000 magnetic nanoparticles. In some embodiments, a nanostructure can comprise from about 100 to about 2,500 magnetic nanoparticles, from about 200 to about 1,000 magnetic nanoparticles, from about 250 to about 900 magnetic nanoparticles, or from about 300 to about 750 magnetic nanoparticles. The number of magnetic nanoparticles per nanostructure can also be expressed as an average number of magnetic nanoparticles per nanostructure, derived from a representative sample of the nanostructures.

The overall size (diameter) of the nanostructure can be tailored for specific purposes. Generally, very large nanostructures can be susceptible to breakage or lysis, whereas very small nanostructures can be difficult to form or can contain an insufficient amount of magnetic nanoparticles. The nanostructures generally have a diameter within the nanometer range; however, nanostructures may also have a diameter above or below the nanometer range. Different methods used to form the nanostructures can result in nanostructures of different diameters or different distribution of diameters. For example, sonication can result in nanostructures having a wide distribution of diameters, whereas an extruder may be used to form nanostructures having more defined diameter ranges. In some embodiments, the nanostructure has a diameter (or an average diameter) of from about 25 nm to about 50,000 nm, from about 50 nm to about 10,000 nm, from about 75 nm to about 5,000 nm, from about 100 nm to about 1,000 nm, or from about 200 nm to about 800 nm. In some embodiments, the nanostructure has a diameter (or an average diameter) of about 200 nm+/−25%, about 400 nm+/−25%, or about 800 nm+/−25%.

The magnetic nanoparticle can be any magnetic nanoparticle capable of encapsulation within membranous material such as cell membrane-derived material, and in which can respond to (be attracted to) a magnetic field. In some embodiments, the magnetic nanoparticle comprises iron (e.g., an iron oxide), but can also comprise other magnetic materials such as nickel or cobalt.

The magnetic nanoparticle has a diameter smaller than that of the nanostructure and thus can be encapsulated therein. Generally, the magnetic nanoparticle can have a diameter of from about 0.1 nm to about 100 nm. In some embodiments, the magnetic nanoparticle can have a diameter of from about 0.5 nm to about 80 nm, from about 1 nm to about 50 nm, from about 5 nm to about 25 nm, or from about 10 nm to about 20 nm. In some embodiments, the magnetic nanoparticle can have a diameter of about 15 nm.

The magnetic nanoparticle can have a surface coating which, in some embodiments, can impart physical properties on the magnetic nanoparticle. For instance, the surface coating can affect the interactions between the magnetic nanoparticle and the cell membrane-derived material or the components therein (e.g., the target membrane protein, membrane lipids, etc.). In some embodiments, the surface coating is a negatively charged surface coating. In some embodiments, the surface coating comprises a moiety selected from tannic acid, a gluconic acid, a citric acid, a glutathione, a quinic acid, a lactobionic acid, a dopamine, a polyacrylic acid, and combinations thereof. In some embodiments, the selected moiety comprises tannic acid, a gluconic acid, a citric acid, or a glutathione.

### Methods of Screening

Also disclosed are methods of screening a sample for a binding agent, the method comprising contacting a sample comprising a binding agent with a nanostructure to form a mixture, the nanostructure comprising a cell membrane-derived material comprising a target membrane protein; and one or more magnetic nanoparticles; wherein the cell membrane-derived material encapsulates the one or more magnetic nanoparticles; and separating the nanostructure and any binding agent bound thereto from the mixture with a magnet.

The method can include any herein disclosed nanostructure or combinations of nanostructures.

In the method, the binding agent can bind to the nanostructure by binding the target membrane protein. The binding agent can bind the target membrane protein in one or more positions of the target membrane protein; however, the method can be suited to identify binding agents which bind at a specific location of a target membrane protein (e.g., a particular binding pocket or receptor region). In some embodiments, the binding agent can specifically bind the target membrane protein. As used herein, the terms “specific binding” or “specifically binds”, in reference to the interaction of a protein or polypeptide with an agent, means that the interaction is dependent upon the presence of a particular structure (e.g., an “epitope”) on the polypeptide or agent. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule.

In some embodiments, binding of the binding agent to the target membrane protein can be determined by comparison to a control. For example, the binding agent may bind a nanostructure comprising a target membrane protein, but does not appreciably bind a substantially similar control nanostructure which does not contain the target membrane protein. In some embodiments, the amount of binding agent which binds a nanostructure comprising a target membrane protein is at least 2-fold, at least 3-fold, at least 4-fold, at least 5-fold, at least 10-fold, at least 20-fold, at least 50-fold, at least 100-fold, at least 500-fold, or at least 1,000-fold more than the amount of binding agent which binds a control nanostructure.

The contacting step of the method generally proceeds for an amount of time sufficient for a binding agent to bind the target membrane protein. In some embodiments, the contacting step is terminated before appreciable non-specific binding occurs. In some embodiments, the contacting step proceeds for at least 1 minute, at least 5 minutes, at least 10 minutes, at least 30 minutes, at least 60 minutes, at least 90 minutes, at least 120 minutes, or at least 180 minutes. In some embodiments, the contacting step is terminated within (and inclusive of) 1 week, 1 day, 12 hours, 6 hours, 3 hours, 2 hours, 1 hour, 30 minutes, 15 minutes, or 10 minutes. The contacting step can be terminated by a variety of methods, including proceeding to the separating step in which the nanostructure and any binding agent bound thereto are separated from the mixture with a magnet.

The sample can be any crude or “dirty” mixture of components (a complex mixture), or can be refined or purified. A particular advantage of the disclose methods is the ability to screen for binding agents present in a complex mixture, for instance a sample comprising numerous unknown components. Upon binding of the binding agent to the target membrane protein, the nanostructure comprising the agent-membrane protein complex can be separated from the remaining complex mixture by use of a magnet. For example, the mixture can be exposed to a magnetic field to attract the nanostructures to the source of the magnetic field (e.g., the magnet), and the nanostructures can then be separated from the remaining components of the mixture.

Thus, in some embodiments, the sample comprises a crude mixture of unknown components, a crude mixture of components in unknown amounts, or any combination thereof. The sample can comprise biological material, non-biological natural material, synthetic material, or any combination thereof. As an example, the sample can comprise an extract of a biological tissue or cell which is unpurified. In a sample comprising a crude mixture of components, the complex sample mixture can contain an array of small molecules and macromolecules in an array of concentrations, some of which may be known and some of which may be unknown. In some embodiments, the sample is from a biological tissue or cell suspected to contain a binding agent that is a pharmacologically active agent. In some embodiments, the sample is an extract of a plant. In some embodiments, the plant is one in which can be combusted (burned) and the smoke contents thereof inhaled (e.g., “smoked”). In some embodiments, the sample can comprise a smoke condensate. As used herein, “smoke condensate” refers to molecules and nanoparticles produced or released by combustion of a plant and present in the smoke generated by such combustion. Smoke condensate can generally be collected by passing the smoke of a combusted plant through a filter, and collecting the particulate matter on the film thereafter (e.g., by extraction with a solvent such as DMSO).

In some embodiments, the sample can comprise a portion of a plant which can be smoked to treat a disease or ailment, such as a neurological disease. In some embodiments, the sample can comprise a portion of a plant which can be smoked to provide stress relief, or to treat sleeplessness, pain, anxiety, or combinations thereof. In some embodiments, the sample can comprise a portion of a plant which can be smoked to achieve “legal high” in one or more jurisdictions. In some embodiments, the sample can comprise a portion of a tobacco plant, a marijuana plant, or combinations thereof. In some embodiments, the sample can comprise a portion of a plant known or suspected to contain compounds which bind to CB1, CB2, opioid receptors (e.g., opioid δ-, κ-, and μ-receptors), monoaminoxidase enzymes (e.g., MAO-A, MAO-B), or combinations thereof. In some embodiments, the sample can comprise Leonotis leonurus (wild dagga), Leonurus cardiaca (motherwort), Eschscholzia californica (California poppy), Nelumbo nucifera (sacred lotus), Magnolia grandiflora (southern magnolia), Peganum harmala (Syrian rue), Banisteriopsis caapi, Desmanthus illinoensis (prairie mimosa), or any combination thereof.

The sample comprises a binding agent, to which the target membrane protein can bind. The binding agent can be known or unknown or be present in known or unknown amounts. The methods are particularly advantageous for screening for an unknown binding agent which binds the target membrane protein. The binding agent can be any small molecule, compound, macromolecule, or molecular complex which can bind the target membrane protein. In some embodiments, the binding agent does not bind in an appreciable amount to other accessible components of the nanostructure (e.g., components present in the cell membrane-derived material). In some embodiments, the binding agent comprises a phytochemical.

Encapsulation of the magnetic nanoparticles within the nanostructure has several advantages. One advantage includes the avoidance of non-specific binding between the magnetic nanoparticles and components of the sample. Some known methods employ magnetic beads attached to a probe used to bind an agent. However, exposure of the magnetic beads to other components in the mixture often leads to interfering, non-specific binding with one or more components of the mixture. This is because numerous components can bind the magnetic beads themselves rather than the probe. Thus, in some embodiments, the method comprises is substantially devoid of non-specific binding between the sample and the magnetic nanoparticles.

Another advantage of encapsulation of the magnetic nanoparticles within the nanostructure includes the provision of a sorting mechanism. After the binding agent binds the target membrane protein, the nanostructure can be attracted to a magnet and collected. This provides a mechanism to sort the nanostructures from the remaining sample, which can include unbound binding agent.

In some embodiments, the method further comprises separating the binding agent from the target membrane protein. Separation of the binding agent can facilitate further identification of the binding agent, for example in analytical tests. Numerous methods can be used to separate the binding agent from the target membrane protein, including but not limited to, addition of a protease, addition of a solvent such as an organic solvent, increasing the ionic strength (e.g., by addition of a salt), addition of a protein denaturant, mechanical agitation (e.g., ultrasonication), boiling, or other known methods. In some embodiments, one or more intervening wash steps can be included between separation of the nanostructures from the mixture and separation of the binding agent from the target membrane protein.

To facilitate reuse of the nanostructures, milder methods to separate the binding agent from the target membrane protein are advantageous. Thus, in some embodiments, the binding agent is separated from the target membrane protein by combining a solvent (e.g., an organic solvent) in an amount up to about 10 percent with the nanostructure. In some embodiments, the solvent is present in a solution comprising the nanostructure in an amount from about 1 percent to about 10 percent, from about 2 percent to about 9 percent, from about 3 percent to about 8 percent, or from about 4 percent to about 7 percent. In some embodiments, the solvent comprises an organic solvent, which for example can be methanol.

An advantage of the disclosed nanostructures and methods of screening using nanostructures is the ability to reuse the nanostructures in further iterations of the method. Thus, in some embodiments, the method can comprise repeating each step reusing the same nanostructure. The further iterations of the method can include the same or different sample. In some embodiments, the nanostructures are used in at least two, at least three, at least four, at least five, or at least ten iterations of the method. The nanostructures can generally be reused as many times as desired so long as the nanoparticle nanostructures continue to function at an acceptable level. In some embodiments, the nanostructure is reusable if the nanostructure binds a known binding agent in an amount which is at least 50% of the amount of the known binding agent bound by a control nanostructure that has not been reused. In some embodiments, the nanostructure is reusable if the nanostructure binds a known binding agent in an amount which is at least 60%, at least 70%, at least 80%, at least 90%, or at least 95% of the amount of the known binding agent bound by a control nanostructure that has not been reused.

The methods can include a step to identify the binding agent. Numerous methods can be used to identify (e.g., determine the structure of) an unknown chemical agent. For example and without limitation, spectroscopic methods can be used, including 1D and 2D nuclear magnetic resonance (NMR), mass spectroscopy (MS), chromatography such as liquid chromatography (LC) and gas chromatography, or combinations thereof.

The binding agent can be further evaluated for pharmacological activity. Thus, in some embodiments, the binding agent can be evaluated in an assay to determine whether the binding agent imparts a response in a biological cell. For example, the binding agent can be evaluated in a cell-based assay to determine if binding of the binding agent to a biological cell elicits a cellular response. In some embodiments, the binding agent can be selected based on the amount of pharmacological activity exhibited in a cell-based assay. For example, in some embodiments, the binding agent is selected if it exhibits a pharmacological activity when present in micromolar amounts. In some embodiments, the binding agent is selected if it exhibits a pharmacological activity when present in submicromolar amounts (e.g., nanomolar or picomolar amounts).

The binding agent can be any small molecule, compound, macromolecule, or molecular complex which can bind the target membrane protein. In some embodiments, the binding agent comprises small molecule, compound, macromolecule, or molecular complex having a pharmacological activity. In some embodiments, the binding agent activates or inhibits a Nuclear Factor Erythroid 2-Related Factor 2 Activation pathway (Nrf-2), a NF-κB pathway, a Wnt-β-catenin pathway, a BDNF pathway, or any combination thereof. In some embodiments, the binding agent activates or inhibits a Wnt-β-catenin pathway or a BDNF pathway. In some embodiments, the binding agent comprises a phytochemical.

A binding agent which binds a target membrane protein can be useful for treating a disease or ailment. The cell type used to supply the cell membrane-derived material can be related to the disease or ailment for which a binding agent having pharmacological activity is useful to treat. An advantage of the disclosed nanostructures and assays is their versatility for screening and discovering from very complex mixtures essentially limitless agents for numerous diseases and thus, the assay can be tailored to screen for agents effective to treat an array of diseases which involve in some way a membrane protein. In some embodiments, the method can identify a binding agent useful for treating a neurodegenerative disease. In some embodiments, the neurodegenerative disease is Alzheimer's disease or Parkinson's disease. In some embodiments, the methods include administering the binding agent to a subject with a disease. The amount administered can vary between subjects, and can be a therapeutically effective amount. In some embodiments, the binding agent administered to a subject is comprised in a pharmaceutical formulation comprising a pharmaceutically acceptable excipient.

Further methods of screening a sample for a binding agent are disclosed in Sherwood J. et al., “Cell-membrane coated iron oxide nanoparticles for isolation and specific identification of drug leads from complex matrices,” Nanoscale, 2019, 11, 6352-6359, the disclosure of which is incorporated herein by reference in its entirety.

### Methods of Making Nanostructures

Also disclosed herein are methods to make a nanostructure comprising contacting a cell membrane-derived material with one or more magnetic nanoparticles to form a mixture, wherein the nanostructure comprises a cell membrane-derived material comprising a target membrane protein; and one or more magnetic nanoparticles; wherein the cell membrane-derived material encapsulates the one or more magnetic nanoparticles.

The methods to make a herein disclosed nanostructure can further comprise expressing a target membrane protein in a biological cell (e.g., by encoding and expressing the protein from an artificial vector such as a plasmid or viral vector). In some embodiments, the methods can further comprise lysing a biological cell to obtain the cell membrane-derived material. In some embodiments, the methods can further comprise extruding the mixture. In some embodiments, the methods can further comprise sonicating (e.g., ultrasonicating) the mixture. In some embodiments, the methods can further comprise confirming the surface-accessibility (and/or the correct orientation) of the target membrane protein by binding a known binding agent to the target membrane protein.

In some embodiments, the methods can comprise contacting the cell membrane-derived material and the magnetic nanoparticles in a weight ratio ranging from about 1:50 to about 1:1,000, from about 1:75 to about 1:800, from about 1:100 to about 1:600, from about 1:150 to about 1:500, or from about 1:200 to about 1:400.

In some embodiments, the methods can comprise contacting the cell membrane-derived material and the magnetic nanoparticles in a ratio to provide a nanostructure comprising from about 50 to about 5,000 magnetic nanoparticles, from about 100 to about 2,500 magnetic nanoparticles, from about 200 to about 1,000 magnetic nanoparticles, from about 250 to about 900 magnetic nanoparticles, or from about 300 to about 750 magnetic nanoparticles. The number of magnetic nanoparticles per nanostructure can also be expressed as an average number of magnetic nanoparticles per nanostructure, derived from a representative sample of the nanostructures.

In some embodiments, the methods can comprise contacting the cell membrane-derived material and the magnetic nanoparticles in a ratio to provide a nanostructure having a diameter (or an average diameter) of from about 25 nm to about 50,000 nm, from about 50 nm to about 10,000 nm, from about 75 nm to about 5,000 nm, from about 100 nm to about 1,000 nm, or from about 200 nm to about 800 nm. In some embodiments, the nanostructure has a diameter (or an average diameter) of about 200 nm+/−25%, about 400 nm+/−25%, or about 800 nm+/−25%.

Further methods of making a nanostructure are disclosed in Sherwood J. et al., “Cell-membrane coated iron oxide nanoparticles for isolation and specific identification of drug leads from complex matrices,” Nanoscale, 2019, 11, 6352-6359, the disclosure of which is incorporated herein by reference in its entirety.

### Examples

To further illustrate the principles of the present disclosure, the following CMNPs preparation and screening examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. These examples are not intended to exclude equivalents and variations of the present invention which are apparent to one skilled in the art. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

### Example 1. Construction of CMNPs

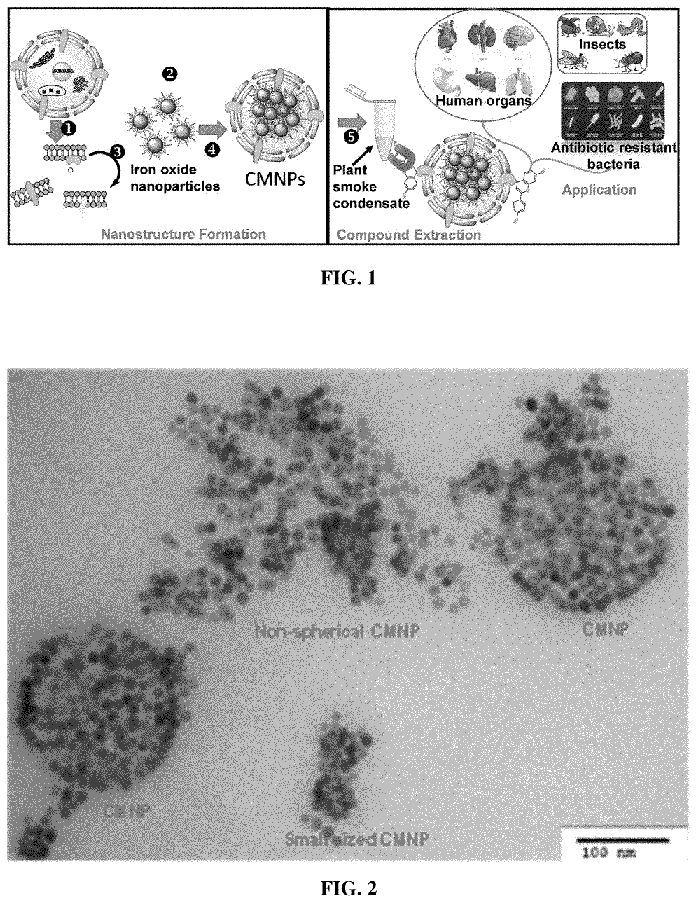

Results disclosed herein describe the feasibility and vast potential of cell-membrane coated nanostructures (also referred to herein as cell-membrane coated nanoparticles; CMNPs) in screening complex matrices for biologically active molecules. Disclosed is a discovery assay based on CMNPs which works as a screening funnel to identify compounds from natural matrices and binding to transmembrane proteins (FIG. 1).

The discovery assay has at least several distinct aspects: (a) CMNPs covered with cell membranes with functional receptors, (b) magnetic nanoparticles incorporated inside CMNPs enabling rapid identification and extraction of binding compounds from complex matrices, (c) a library of potential binding ligands, for instance plant smoke condensates as an example of a complex mixture.

Cell membranes with any functional receptors can be used to encapsulate iron oxide magnetic nanoparticles. The following are selected examples of transmembrane proteins that can be used for the preparation of CMNPs: tropomyosin receptor kinase B (TrkB), Frizzled-1 (FZD1), nicotinic receptors, TRP channels, olfactory receptors, etc. The encapsulated nanoparticles can be iron oxide magnetic nanoparticles varying in size and with surface chemically modified by different molecules. The encapsulation process can be performed either using sonication or extrusion or both steps. When both steps are used to form nanostructures, the sonication step typically precedes the extrusion step.

The assay is fundamentally different from traditional plate-based assays, which require pre-defined compound libraries. The disclosed assay facilitates “fishing out” pharmacologically active compounds which bind to transmembrane proteins from complex matrices. Thus, the assay has a substantial impact on the discovery process of biologically active compounds from complex matrices, which can identify and extract new leads from complex samples. The assay can be readily adjusted to any transmembrane protein target from any membrane of any biological organism.

The assay comprises a core of iron oxide nanoparticles and a shell of cell membranes with functional transmembrane receptors (FIG. 1). The presence of functional transmembrane proteins facilitates selective identification of binding compounds in complex matrices. Encapsulation of magnetic nanoparticles enables rapid separation of compounds targeting transmembrane proteins with minimal nonspecific binding.

Several types of CMNPs varying in size and containing magnetic nanoparticles having different surface chemistries can be formed and evaluated. Several parameters can affect performance of the assay: a) size of the CMNPs (effects on activity and stability); (b) surface chemistry of nanoparticles (encapsulation efficiency and CMNP stability); and (c) techniques of cell membrane fragment preparation. Preparation of CMNPs involves several steps (FIG. 1), including 1) preparation of cell membrane fragments with functional transmembrane receptors, 2) synthesis and surface functionalization of magnetic iron oxide nanoparticles, and 3) preparation of CMNPs. Each step contributes to enhancing bioactivities of CMNPs.

As immobilization of functional transmembrane receptors present on the surface of cell membrane fragments is important for assay performance, the buffer conditions for preparation of cell membrane fragments should be carefully selected (buffer types, salts, protease inhibitors, etc.). For magnetic iron oxide nanoparticles, several parameters should be controlled to ensure effective magnetic separation and complete membrane encapsulation. First, the size of the magnetic nanoparticles must be large enough (>5 nm) for quick magnetic response and small enough (<25 nm) to avoid aggregation due to magnetic interactions (Bao et al., J. of Mat. Sci. 2016; 51(1):513-53). Therefore, nanoparticles in the size range of 6-25 nm should be selected. Further, the surface coatings of the magnetic nanoparticles directly interface with the interior parts of the cell membrane fragments and influence the cell membrane coverage. Previous studies on red blood cell membrane coated polymeric nanostructures suggested that negatively charged surfaces facilitated cell membrane coverage and positively charged surfaces formed aggregates of cell membrane fragments and nanoparticles (Luk et al., Nanoscale. 2014; 6(5):2730-7; Jang H S., Molecules. 2017; 22(12)). Here, any molecules that will lead to a negatively charged surface can be used as capping molecules to functionalized the iron oxide nanoparticles. Specific examples can be small molecules, such as gluconic acid, citric acid, lactobionic acid, quinic acid, tannic acid, dopamine, polymers, polyacrylic acid, alginate, etc., or peptides and proteins, such as glutathione, aspartic acid, etc.

Preparation of Membrane Fragments and Magnetic Nanoparticles.

As an example application of the technology, CMNPs carrying transmembrane with nicotine receptors.

Cell membrane fragments were prepared from selected cell lines overexpressing nicotine transmembrane receptors (Ciesla et al., J Chromatogr A. 2016; 1431:138-44). Briefly, 1×10⁷ cells were suspended in Tris-HCl buffer (pH 7.4, 50 mM) supplemented with salts and protease inhibitors. The suspension then was homogenized using Dounce glass homogenizer. The mixture was centrifuged at low speed to remove cell debris and organelles. The remaining supernatant was centrifuged at high speed, and the resulting pellet of cell membranes was used to prepare CMNPs.

Magnetic nanoparticles having a diameter of about 15 nm were used as an example. At such a size, the nanoparticles are superparamagnetic (on/off with the magnetic field), but below the ferromagnetic limit (avoid aggregation due to magnetic interactions). Based on previous studies (Luk et al., Nanoscale. 2014; 6(5):2730-7; Jang H S., Molecules. 2017; 22(12)), negatively charged surfaces are preferred for complete cell membrane coverage on nanoparticles. Several biologically relevant surface coatings, such as tannic acids (high degree of phenol groups), gluconic acid (sugar), citric acid (carboxylic groups) and glutathione (peptide zwitterions) were tested. These molecules provide negatively charged surfaces for iron oxide nanoparticles. However, the different functional groups have different affinities with the inner parts of the cell membrane fragments, thereby allowing the study of the surface chemistry effects on CMNP.

CMNP Formation.

CMNPs were prepared using two different methods: extrusion and ultra-sonication. Both methods have been applied to create cell-membrane coated nanoparticles for drug delivery or targeting (Gao et al., J. Drug Targeting. 2015; 23(7-8):619-26; Narain et al., Nanomedicine. 2017; 12(21):2677-92; Kroll et al., Bioconjugate Chem., 2017; 28(1):23-32; Guo et al., Small. 2018; 14(18); Hu et al., Nature. 2015; 526(7571):118-21).

The extrusion method involves passing the mixture of cell membrane fragments and nanoparticles through porous membranes with defined sizes (200, 400, and 800 nm) using an Avanti extruder. In contrast, for the tip sonication method, the cell membrane fragments and nanoparticle solution can be sonicated using tip sonication, where the amplitude, frequency, and duration are optimized to maximize the formation of CMNPs and minimize the protein denaturation. For both methods, several factors affect the formation of CMNPs. First, the membrane to magnetic nanoparticle ratios influence the membrane coverage on the surface of magnetic nanoparticles. Complete coverage is important for bioactivity evaluation. The theoretical ratio can be estimated using the original cell size, cell concentration, and nanoparticle size. For instance, SH-SY5Y cells are roughly 12 μm in diameter, and adhesive cells only have two major surfaces (top and bottom), thus, the total cell membrane surface area (A) of 10⁷ number cells is calculated according to Formula 1 below:

A=10⁷×2πR², wherein R=1 μm in this example. Formula 1

If CMNPs having a diameter of about 200 nm were targeted, the rough number of CMNPs can be estimated by Formula 2 below:

$${N}_{CMNP} = \frac{{10}^{7} \times 2 π {R}^{2}}{4 {π}^{2}} , where r = 100 nm . \qquad Formula 2$$

The number of CMNPs was thus calculated to be about 10⁹-10¹⁰ depending on the membrane recovery. The CMNP concentration can also experimentally evaluated using dynamic light scattering (DLS) with an internal standard (Xie et al., Nanomed.-Nanotech. Biol. Med. 2007; 3(1):89-94), because the intensity scattered light is proportional to the number of CMNPs (Shang et al., Chem. Soc. Rev. 2014; 43(21):7267-78). Bovine serum albumin protein and polymer beads can be used as internal standards to quantify the amount of CMNPs, using methods described in Xie et al., Nanomed.-Nanotech. Biol. Med. 2007; 3(1):89-94. Additionally, the minimal ratio of the cell membrane to iron oxide nanoparticles can be estimated by CMNP volume divided by individual nanoparticle volume according to Formula 3 below:

$$Ratio = \frac{{r}_{CMNP}^{3}}{{r}_{NP}^{3}} \qquad Formula 3$$

The amount of magnetic nanoparticles should be smaller than this theoretically calculated value to obtain full coverage of the cell membranes. During the CMNP formation process, the effects of buffer conditions, nanoparticle surfaces, membrane-to-nanoparticle ratios, and methods of preparation can be studied on CMNP size, membrane coverage, and bioactivity.

### Example 2. Evaluation and Characterization of CMNPs

The characterization of the CMNPs involves several levels of confirmation and verifications, including confirmation of the CMNP formation, estimation of CMNP concentration, presence of surface transmembrane receptors, correct orientations of the surface receptor, biological activity of the surface receptors and effectiveness of CMNPs in compound fishing.

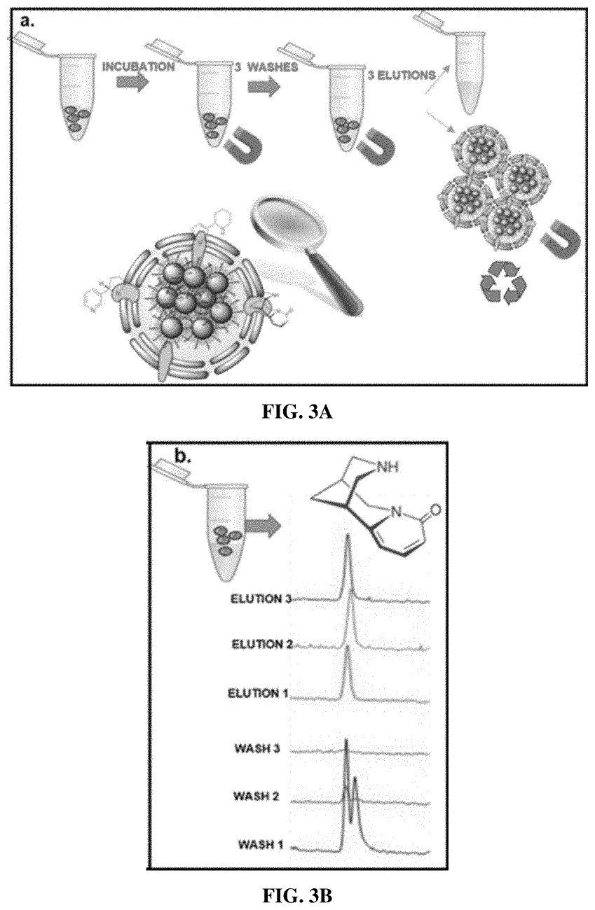

The size, size distribution, and membrane surface coverage of the CMNPs can be studied by transmission electron microscopy (TEM). Cell membranes and iron oxide nanoparticles have large differences in electron densities and appear as different contrasts in TEM images. Generally, membrane shells are light gray circles or barely seen while iron oxide nanoparticles are darker. The hydrodynamic size and size distribution in solution and surface charges can be studied using DLS. CMNP formation is confirmed using TEM, indicated by the spherical groups of iron oxide nanoparticles (small black dots) presented in FIG. 2. Depending on the number of magnetic nanoparticles encapsulated inside, some of the CMNPs can be either non-spherical or small in size. Because of the light electron densities of carbon-based molecules, membrane shells were barely seen.

The estimation of the CMNP concentration was done mainly by theoretical calculation based on the size of the CMNPs, size of the cells and concentration of the cells. The specific calculation process was described in example 1.

The yield consistency of cell membrane fragments obtained in the cell lysis step can affect the ratio of membrane fragments to magnetic nanoparticles. Thus, buffer types and experimental conditions can be standardized to facilitate reproducible cell membrane recovery. Prior to fishing experiments, magnetic separation of CMNPs can be performed to remove nonmagnetic CMNPs (e.g., those with an insufficient number of nanoparticles for magnetic separation).

Biological activities of the CMNP assay can be evaluated by the presence and orientation of the transmembrane receptors (right-side-out) and the compound(s) binding to the receptors. Receptor orientation and compound binding ability are important for the CMNP assay. The presence of transmembrane receptors can be studied by SDS-PAGE. Protein profiles of the extruded membrane fragments and CMNPs can be compared. Orientation of transmembrane receptors (right-side-out) can be verified using flow cytometry. Specifically, fluorescence labeled antibodies can be used to label the surfaces of the CMNPs followed by magnetic separation. Then, flow cytometry analysis can be performed. Only the receptors with correct orientation will interact with the antibody, and thus the percentage of the correct (right-side-out) samples can be obtained. Successful immobilization of transmembrane proteins can also be confirmed using confocal microscopy. Additionally, nanotechnology techniques can be used to directly visualize transmembrane receptors. In brief, ligand standards with known binding to tested cell surface receptors can be conjugated on gold (Au) nanoparticles and then mixed in the CMNP assay. Places with membrane receptors directly bound the ligand on Au nanoparticles. Because of the much higher electron density of Au compared to iron oxide, Au nanoparticles appeared much darker on TEM grids, which facilitated visualization of transmembrane receptors on CMNP surfaces.

### Example 3. The Use of Nanostructures in Screening Complex Samples for Biologically Active Compounds (Ligand Fishing)

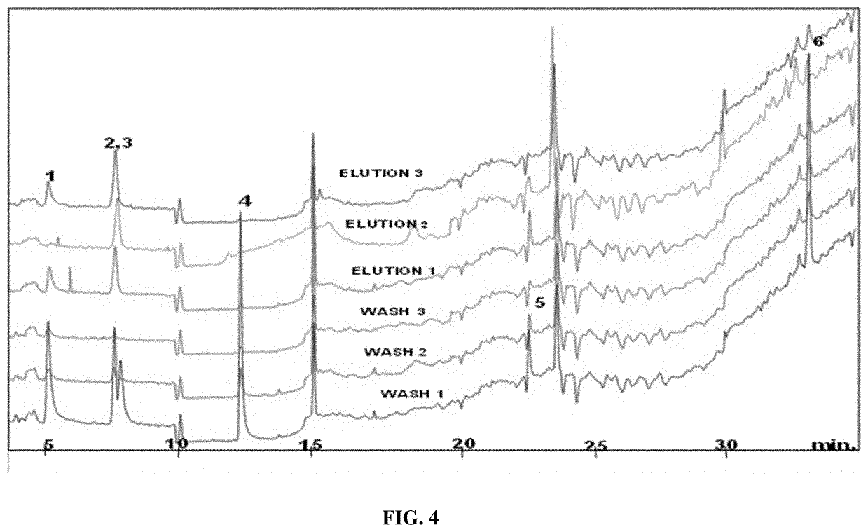

Fishing experiments can be used to evaluate the CMNP assay. As an example, CMNPs were constructed with immobilized nicotinic receptors. Parameters include selectivity, detection limit, stability, and reuse of CMNPs. The assay or fishing experiment (FIG. 3A) involves magnetic separation of CMNPs with bound compounds after 20 min incubation in an artificial mixture of known binders and nonbinders. After 3 washes with buffer, receptor-bound compounds are released during the elution process. Eluted compounds can be analyzed by high-performance liquid chromatography coupled with diode-array detection and electrospray ionization tandem mass spectrometry (HPLC-DAD-MS).

As another example, cytisine is a known ligand of nicotinic receptors and can be used to demonstrate ligand capture, elution, and subsequent identification. FIG. 3B shows results for cytisine ligand released from CMNPs containing nicotinic receptors.

Selectivity and detection limits of CMNPs are important for compound identification in the fishing step because natural compound mixtures, especially secondary metabolites, vary in pH, polarity and compound concentrations and have large chemical and structural diversity (Mathur et al., Biomedical Reports. 2017; 6(6):612-4). Some active compounds may be present at a very low concentration in the analyzed mixture.

Stability of the CMNPs is also an important parameter for future use, application and storage. All previous studies on cell-membrane coated nanoparticles focused on drug delivery or tumor targeting (Krishnamurthy et al., Nanoscale. 2016; 8(13):6981-5). Therefore, reuse has not been previously considered. The disclosed CMNPs and assay using CMNPs are firsts for drug identification assays in which reuse is feasible, which reduces costs of drug discovery, an important parameter in industrial settings.

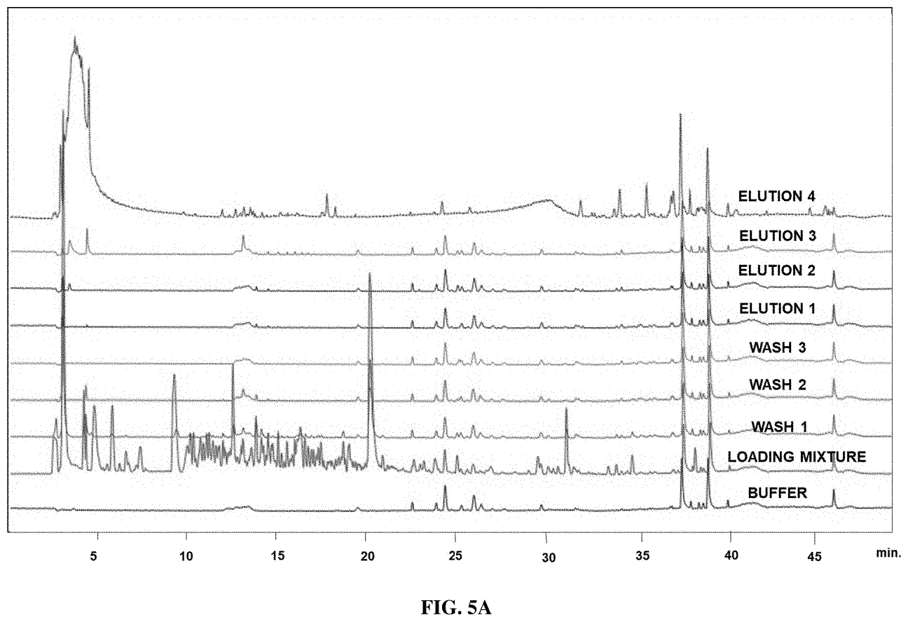

Screening experiments can be conducted with samples of various level of complexity (artificial mixtures, natural extracts with known binders). First, artificial mixtures of known binders and non-binders can be used to evaluate selectivity. In a particular example, CMNPs containing nicotinic receptors were prepared. An artificial equimolar (100 nM) mixture of known binders and non-binders was also prepared. Known binders included known nicotinic receptor binders: nicotine (#1), anabasine (#2), cytisine (#3) and non-binders: butyrylcholine iodide (#4), berberine (#5), warfarin (#6) and caffeic acid (#7). First, the CMNPs (˜10⁹) were placed in 0.5 mL artificial mixture and incubated. CMNPs were subsequently washed three times with ammonium acetate buffer. After washing, CMNPs were eluted with a buffer:methanol (9:1, v/v) mixture. In both the washing and elution steps, CMNPs were separated from supernatant using a magnet. Results of screening experiments for CMNPs with immobilized nicotinic receptors and an artificial mixture are presented in FIG. 4. CMNPs selectively retained the known binders (nicotine, anabasine, cytisine), which were all freed during the elution steps. The absence of the known non-binders (butyrylcholine, berberine, caffeic acid) suggested their removal during the washing steps. Results also indicate the lack of nonspecific binding of cytisine, nicotine and anabasine (data not shown), which was assessed in the fishing experiments performed with CMNPs prepared using parental HEK cell line not expressing nicotinic receptors.

Concentrations of all artificial mixture compounds can be set at 100 nM. Normally, the concentration of secondary metabolites in natural mixtures varies from nano to micromolar range. Selective binding to transmembrane receptors can be confirmed by comparing the results of screening experiments obtained with CMNPs with the immobilized targeted receptors and CMNPs prepared with cell lines not expressing the receptors. Since the CMNPs(+) and CMNPs(−) essentially differ only in the availability of the receptors (FZD1/TrkB), any differences in binding of a compound to the CMNPs(+) relative to the CMNPs(−) is due to specific interactions with the investigated receptors.

To assess the performance of CMNPs in natural compound mixtures, a known binder can be added to a selected plant extract at 100 nM concentration. Experiments can be repeated for, e.g., five defined binders to confirm selectivity for different known binding ligands. Selectivity of the CMNP can be confirmed if the nanoparticles with the immobilized receptors could “fish out” only binders from an equimolar artificial mixture and natural compound mixture. Nonspecific binding should be lower than 5%, confirmable by HPLC-MS analysis.

Detection limits of CMNPs can be evaluated using both artificial and natural product mixtures. For artificial mixtures, the concentration of nonbinders can be, e.g., 100 nM, while the binder concentration can vary (e.g., 1, 10, 25, 50, 100 nM). The detection limit of CMNPs can be evaluated by showing the lowest concentration of the known binder to be detected as binding to transmembrane proteins. Then, natural compound mixture with the known added binder of different concentrations (0, 10, 25, 50, 100 nM) can be used to study the detection limit in natural compound mixtures.

### Example 4. Stability and Reuse of Nanostructures

Stability evaluation covers structural integrity and bioactivity of the transmembrane receptors. The stability of CMNPs can be evaluated under various conditions, such as: (a) buffers at various pH (6, 7, 8, and 9), because natural compound mixtures vary greatly in pH and CMNPs can be generated to survive some or all of these conditions for the duration of the tests (incubation and fishing experiments, roughly 2 hours). Therefore, CMNP samples can be incubated in Tris buffer at different pH for two hours at room temperature. (b) Different temperatures (4, 25, and 37° C.), a set of temperatures can be selected to represent the preparation, ligand fishing and storage conditions. For each condition, CMNP structure integrity can be visualized under TEM and tested using DLS for hydrodynamic size and surface charge. Information can be obtained on factors affecting CMNP stability, such as size of CMNPs, surface chemistries of magnetic nanoparticles, and methods of CMNP preparation. To assess biological activities of CMNPs under different conditions, the detection limits and selectivity can be evaluated and compared. Loss of biological activity can alter the selectivity and the detection limit.

Reuse of the CMNP assay can be evaluated by repeating the same experiments using the previously described artificial and natural compound mixtures. Detection limit values can be used to determine the reusability of the CMNPs. When a 50% decrease in detection limit is observed, the prepared batch of CMNPs is considered no longer reusable.

### Example 5. Screening of Smoke Condensates Against CMNPs Containing Nicotinic Receptors

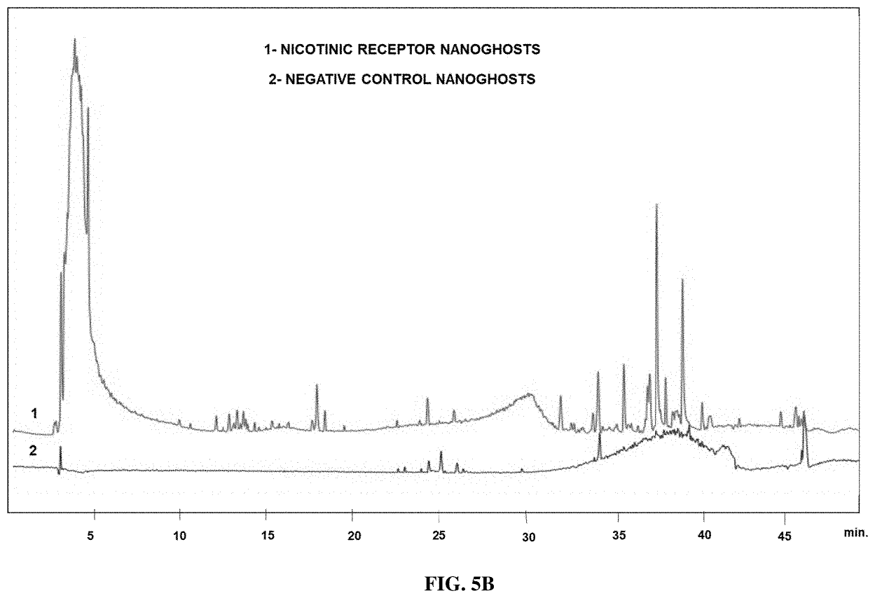

As an example, CMNPs containing nicotinic receptors were used to screen a different set of potential binders in a crude mixture: that of tobacco smoke condensates (FIG. 5). The results obtained with nicotinic receptor CMNPs were compared with those obtained for negative control CMNPs. Negative control CMNPs were prepared from the parental HEK 293 cell line that does not express nicotinic receptors. Therefore, the only difference between nicotinic and negative control CMNPs is the absence of nicotinic receptors in control CMNPs. After three washes with ammonium acetate buffer, the CMNPs were eluted using first ammonium acetate buffer:methanol mixture (9:1, v/v; elutions 1-3), and then ammonium acetate buffer methanol mixture (1:9, v/v, elution 4). FIG. 5 shows that numerous compounds were freed from the nicotinic CMNPs during elution 4. Each of these compounds were not present in elution 4 profile of negative CMNPs, indicating the compounds likely specifically bound nicotinic receptors.

### Example 6. Screening Plant Smoke Condensates for Pharmacologically Active Compounds Using CMNPS with Immobilized TrkB and FZD1 Receptors

To address the challenges of identifying new neurotherapeutic agents from complex natural matrices, one must address the lack of suitable screening assays. While substantial advancement in studying the effects of intermittent fasting and aerobic exercise on neuronal protection has been made (Mattson et al., Nat Rev Neurosci. 2018; 19(2):63-80), the progress in studying neuroprotective phytochemicals is less successful. One main limitation is the complexity of natural products and lack of suitable tools to identify biologically active compounds in complex matrices.

This hurdle can be addressed in the early stages of drug discovery processes by the disclosed CMNP assay. The assay uses magnetic nanoparticles encapsulated inside cell membrane material expressing targeted functional receptors (e.g., TrkB or FZD1).

As it relates to neurodegenerative diseases as an example application of this technology, the disclosed example CMNP assay addresses at least two major challenges in drug discovery: (1) the lack of effective treatment of neurodegenerative diseases such as Alzheimer's disease, and (2) the absence of drug discovery assays suitable to screen complex matrices.

As an example, the assay can be used to identify compounds which stimulate two pathways: BDNF and canonical WNT-β-catenin pathways. Compounds binding to transmembrane TrkB and FZD1 receptors can stimulate BDNF and WNT-β-catenin pathway and increase cell resilience. Interestingly, a natural compound, 7,8-dihydroxyflavone was previously identified as a TrkB agonist and potential drug lead to treat several BDNF-implicated diseases (Liu et al., Transl Neurodegener. 2016; 5:2).

Disclosed herein is an assay for drug lead identification from complex natural matrices. Additionally, the assay can focus on, as an example, neuroprotective stress response cellular signaling pathways. For instance, the BDNF signaling pathway and canonical WNT-β-catenin pathway can be targeted, both of which are hypothesized to be adaptive cellular pathways and thought to be a part of the stress response system that evolved to protect neurons from different forms of biological stress (Mattson M P., Dose-Response. 2014; 12(4):600-18).

As yet another example, the disclosed assay can be used to discover compounds from plant smoke condensates with potential as neurotherapeutic agents. Plant smoke condensates are crude mixtures of compounds, some of which may bind to and activate neurological pathways. As examples, the transmembrane receptors TrkB and FZD1 are involved in activating the Wnt-β-catenin and/or BDNF pathways. Plant smoke condensates may contain compounds which bind to TrkB or FZD1 and therefore activate Wnt-β-catenin and/or BDNF pathways. Smoke condensates may be an excellent source of potential new drug leads (Mohagheghzadeh et al., J Ethnopharmacol. 2006; 108(2):161-84). Secondary metabolites and their degradation products present in smoke can penetrate the blood brain barrier and bind to multiple receptors and exert pharmacological effects. Thus, screening of plant smoke condensates in particular can increase the chances of identifying compounds that penetrate the blood brain barrier and bind to transmembrane receptors (e.g., TrkB or FZD1).

Selection of plants for smoke condensates can be based on their historical reputation for smoking practice and data generated on neuronal receptors and enzymes in previous studies. Among these, Leonotis leonurus, Eschscholzia californica, Nelumbo nucifera, Magnolia grandiflora, Peganum harmala and Banisteriopsis caapi have demonstrated significant binding to CB1, CB2 and/opioid receptors, as well as monoaminoxidase (MAO) inhibitory and antioxidant activities.

Compounds identified as binding to CMNPs containing TrkB or FZD1 receptors can be isolated (e.g., by using preparative high-resolution liquid chromatography, centrifugal thin-layer chromatography, TLC/HPLC/UPLC-MS/GC-qToF), and compound structures can be elucidated by spectroscopic techniques (e.g., high-resolution mass spectrometry, liquid chromatography/mass spectrometry, nuclear magnetic resonance spectroscopy such as 1D and 2D NMR, and GC/Q-ToF). Activity of isolated compounds can be verified in cell-based assays. In vivo drug disposition such as volume of distribution, drug efficiency, tissue/plasma partition can be assessed using Bio-Mimetic Chromatography (BMC) models (Valko K L. J Pharm Biomed Anal. 2016; 130:35-54).

Thus, the assay works as a testing funnel, narrowing down hundreds of compounds present in a complex matrix to compounds specifically binding to a transmembrane receptor. By this example method, new drug leads for the prevention and treatment of neurodegenerative diseases such as Alzheimer's disease can be identified.

### Example 7. Using CMNPs Containing Olfactory and Taste Receptors from Insect Cells to Screen Microbial Metabolites

Other example transmembrane receptors having important functions include olfactory and taste receptors, which can also be incorporated into CMNPs and screened against a crude mixture of potential binding compounds. Olfactory and taste receptors are expressed in sensory organs to recognize chemical signals from the environment. However, little is known about the molecular basis of chemical signaling between gut microbiota and host organism cells. Interestingly, ectopic olfactory and taste receptors are expressed in cells in a variety of animal host organs including the kidney, brain, heart, and gut (Flegel et al., PLoS One. 2013; 8(2):e55368). Functional ectopic olfactory receptors are present in the kidney and respond to high concentrations of short-chain fatty acids produced by gut microbiota (Pluznick et al., PNAS USA., 2013; 110(11):4410-5). Activation of ectopic olfactory receptors expressed in liver reverses obesity (Crunkhorn S., Nat Rev Drug Discov., 2017; 16(12):826-7). In addition, ectopic olfactory receptors play a role in the progression of prostate cancer (Abaffy et al., Front Oncol. 2018; 8:162). Unfortunately, most putative ectopic olfactory receptors remain functionally uncharacterized and their roles in the host remain unknown.

Olfactory receptors in nasal epithelium or antennal sensory tissues play a crucial role in chemical communication with the surrounding environment. Ectopic olfactory receptors can play a similar role in recognizing chemical signals produced by gut microbiota, an internal ecosystem.

Known ligands for olfactory receptors expressed in olfactory receptor neurons are small, lipophilic molecules. Small lipophilic gut microbial metabolites can be ligands for ectopic olfactory receptors expressed throughout the host organism. Due to their lipophilic nature, such ligands may require protein transporters to be distributed in predominantly aqueous environments such as the animal's circulatory system. Interestingly, odorant binding proteins have been found in tissues unrelated to olfaction, such as the brain, accessory sex glands, and hemolymph of Drosophila melanogaster and other insects (Graham et al., Gene. 2002; 292(1-2):43-55). Humans and other vertebrates also possess odorant-binding proteins that transport odorant molecules to olfactory receptors in the nasal epithelium (Tegoni et al., Biochim Biophys Acta. 2000; 1482(1-2):229-40).

Since little is known about the function of ectopic olfactory receptors, there are no assays for identifying ligands produced by gut microbiota. Microbial metabolites may be ligands for ectopic olfactory receptors, and are present in complex matrices, which hinders identification of biologically active compounds. However, the disclosed bioassays facilitate identification of biologically active compounds present in complex natural matrices.

The disclosed bioassay can be used to identify ligands of an olfactory receptor, for example, ectopic olfactory receptors of the fruit fly Drosophila melanogaster. A Drosophila model of metabolic syndrome can be used to identify ectopic olfactory receptors expressed in Drosophila gut and odorant binding-proteins. The identified ectopic olfactory receptors and odorant-binding proteins can be overexpressed in Drosophila and gut tissues can be used to prepare the assays using CMNPs.

These tools and bioassays answer an important question in current biological science: how gut microbiota communicate with host cells. Identification of microbial chemical signals facilitates development of new therapeutic strategies based on targeted dietary or pharmacological intervention to prevent and treat numerous diseases, for example diabetes, depression, neurodegenerative illnesses or some forms of cancer. The disclosed methods are applicable to studies focused on identifying volatile, small, and low abundance ligands.

### Example 8: Immobilization of TrkB Receptors on CMNP

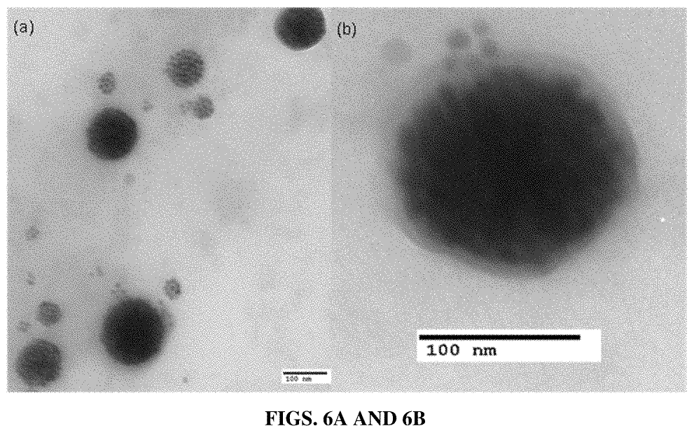

Functional TrkB receptors (example of tyrosine kinase receptors) were immobilized on nanoparticles using the CMNPs technology described herein. Neuroblastoma SH-SY5Y cell line stably overexpressing TrkB receptors were used. Cell membrane fragments and nanoparticles used in the preparation of CMNPs were obtained using the previously optimized protocols, as described for the nicotinic receptors. FIG. 6 shows successful assemble of CMNPs with TrkB receptors on their surface.

The immobilized TrkB receptors were used in fishing experiments using equimolar (100 nM) mixture of known binder (7,8-dihydroxyflavone) and non-binders (nicotine, caffeic acid, rutin) to demonstrate their activity. The fishing experiments were performed using the procedure previously optimized and described herein for nicotinic receptors. Washing and elution steps were performed using solvents previously reported for nicotinic receptor experiments. The difference between nicotinic and TrkB receptor experiments was with the type of artificial mixture components.

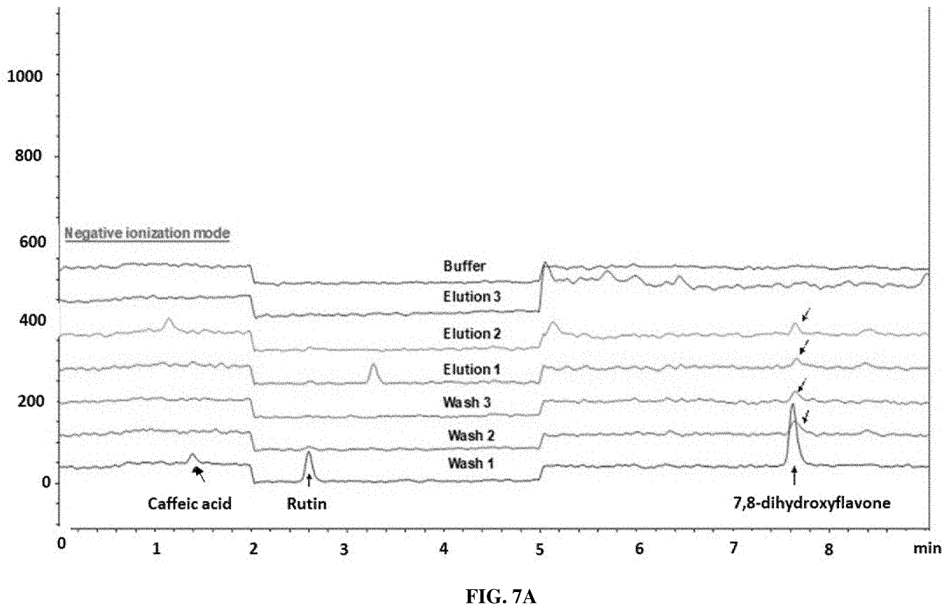

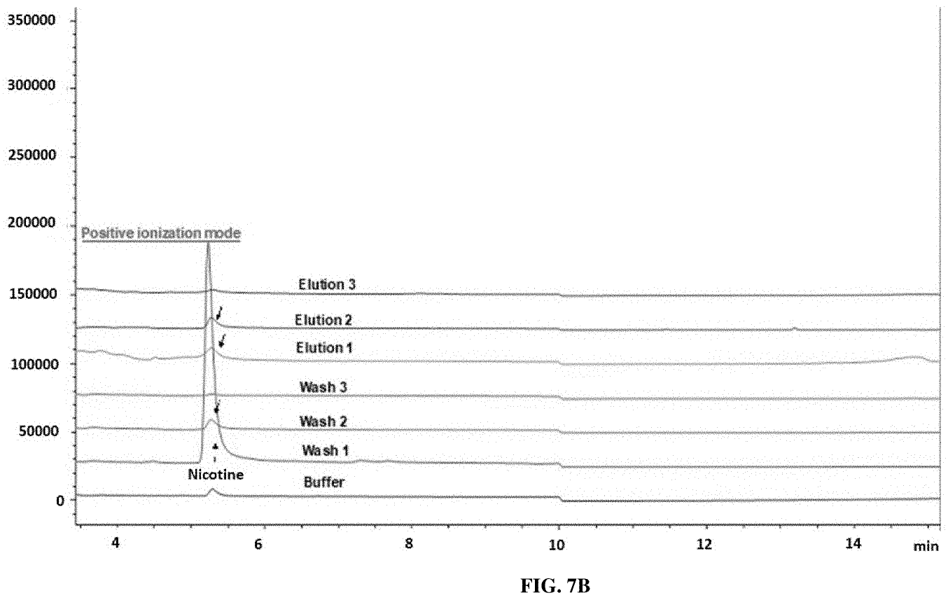

The experiments showed that CMNPs with the immobilized TrkB receptors retained known binder (7,8-DHF), while not retaining the nonbinders, as shown in FIG. 7A. It was further observed that CMNPs with TrkB receptors retained small amount of nicotine (FIG. 7B). Cell membrane fragments used for the preparation of CMNPs were obtained from neuroblastoma cell line that also expresses nicotinic receptors. The retention of nicotine was likely caused by the presence of nicotinic receptors.

The use of negative control CMNPs prepared with the parental SH-SY5Y cell line without TrkB receptors allowed discernment between specific and non-specific interactions. The known binder, 7,8-dihydroxyflavone interacted only with CMNPs with TrkB receptors, while no interaction was observed for negative control CMNPs. Nicotine interacted both with CMNPs with and without TrkB receptors since both cell lines expressed nicotinic receptors. Compounds interacting only with CMNPs expressing TrkB receptors and not interacting with negative control CMNPs are specifically binding with TrkB receptors.

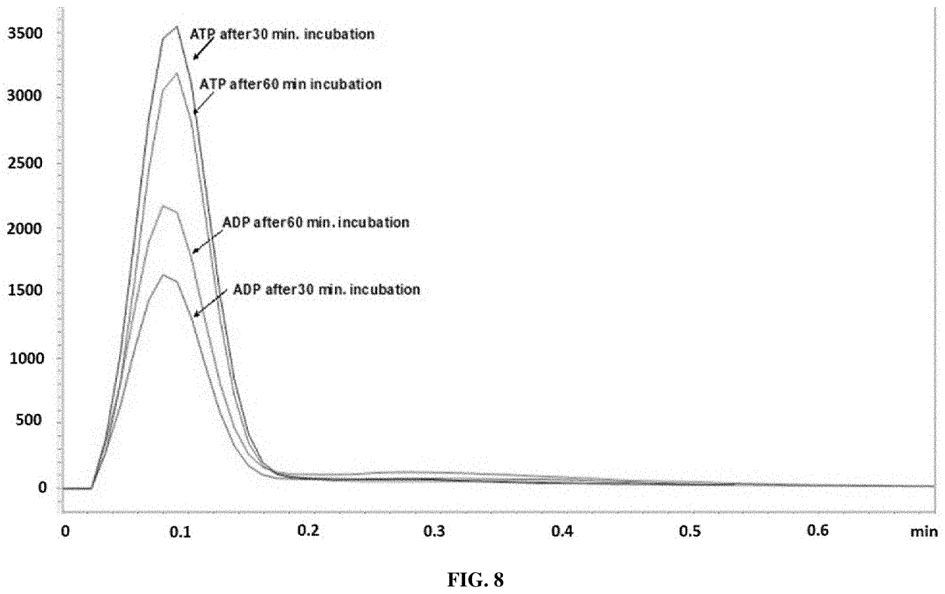

To further test the activity of the immobilized TrkB receptors, additional experiments that focused on determining the ability of the immobilized receptors to convert ATP to ADP were performed, a process commonly performed by all types of tyrosine kinase receptors. Tyrosine kinases, when active, convert ATP to ADP as they use phosphate groups to phosphorylate targeted proteins in the process of signal transduction inside a cell. To this end, CMNPs were incubated with TrkB receptors together with 5 mM of ATP and 100 μM of known activator 7,8-dihydroxyflavone for 30 and 60 min at the temp. 37° C. The progressing conversion of ATP to ADP as presented in FIG. 8 was observed.

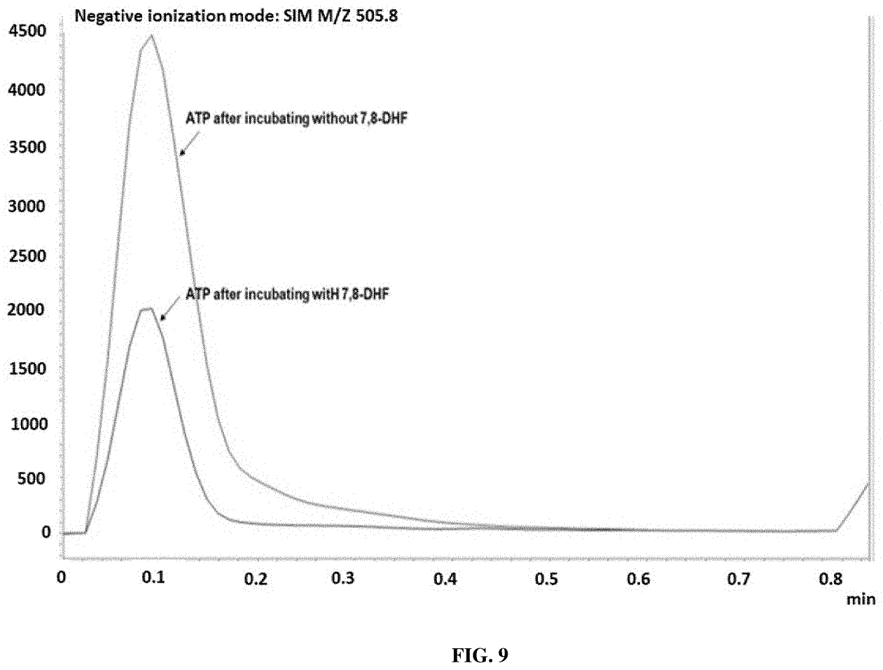

The presence of an activator (7,8-DHF) has been shown as important to observe the ATP-converting activity of the TrkB receptor. CMNPs incubated with 5 mM ATP but without the activator were observed not to convert ATP to ADP. FIG. 9 shows the ATP level in the mixture after incubating CMNPS with and without the activator.

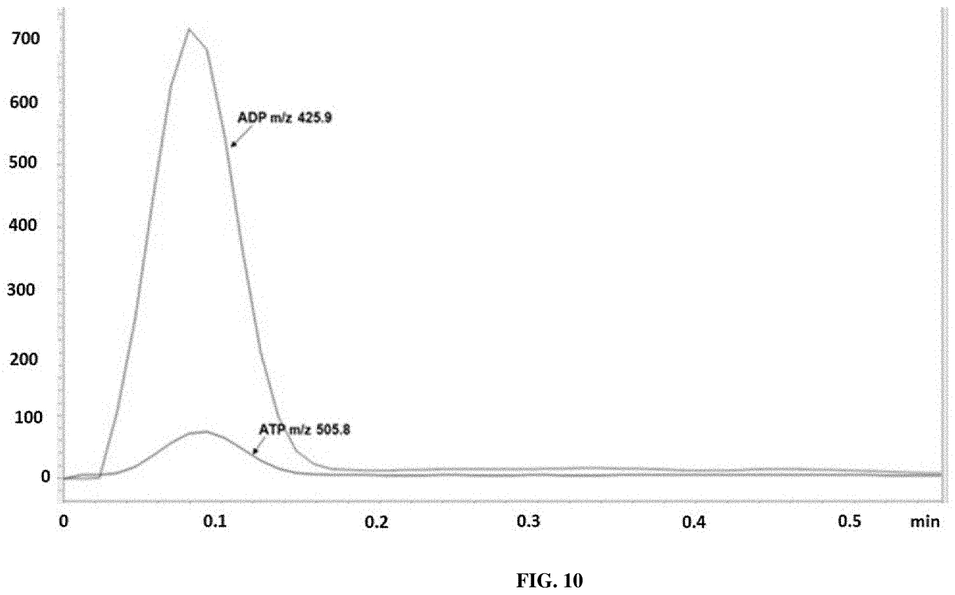

24-hour incubation of CMNPs with a mixture containing 5 mM ATP and known TrkB activator 7,8-dihydroxyflavone (100 μM) at the temperature of 4° C., resulted in almost complete conversion of ATP to ADP, as presented in FIG. 10.

Also performed were experiments to test the possible involvement of nanoparticles (structures inside CMNPs) on the ATP-converting activity of TrkB receptors. The nanoparticles themselves did not lead to the conversion of ATP to ADP when incubated with of ATP (5 mM) and an activator 7,8-dihydroxyflavone (100 μM).

Publications cited herein are hereby specifically incorporated by reference in their entireties and at least for the material for which they are cited.

It should be understood that while the present disclosure has been provided in detail with respect to certain illustrative and specific aspects thereof, it should not be considered limited to such, as numerous modifications are possible without departing from the broad spirit and scope of the present disclosure as defined in the appended claims. It is, therefore, intended that the appended claims cover all such equivalent variations as fall within the true spirit and scope of the invention.

What is claimed is:

1. A method of screening a sample for a binding agent, the method comprising:
a) contacting the sample comprising the binding agent with a nanostructure to form a mixture,
wherein the nanostructure comprises one or more magnetic nanoparticles and a cell membrane-derived material comprising a target membrane protein; and wherein the cell membrane-derived material encapsulates the one or more magnetic nanoparticles; and
b) separating the nanostructure and any binding agent bound thereto from the mixture with a magnet.

2. The method of claim 1, wherein the sample comprises a biological extract.

3. The method of claim 2, wherein the biological extract is from a plant.

4. The method of claim 1, wherein the sample comprises a plant smoke condensate.

5. The method of claim 1, wherein the cell membrane-derived material is from a human cell, an animal cell, a plant cell, an insect cell or a bacterial cell.

6. The method of claim 1, further comprising separating the binding agent from the target membrane protein.

7. The method of claim 1, further comprising identifying the binding agent bound to the target membrane protein.

8. The method of claim 1, wherein the binding agent is a phytochemical.

9. The method of claim 1, further comprising determining a pharmacological activity of the binding agent in a cell-based assay.

10. The method of claim 1, further comprising administering the binding agent to a subject with a disease.

11. A nanostructure comprising:
a cell membrane-derived material comprising a target membrane protein; and
one or more magnetic nanoparticles;
wherein the cell membrane-derived material encapsulates the one or more magnetic nanoparticles.

12. The nanostructure of claim 11, wherein the nanostructure comprises the cell membrane-derived material and the magnetic nanoparticles in a weight ratio ranging from about 1:100 to about 1:600.

13. The nanostructure of claim 11, wherein the nanostructure comprises from about 200 to about 1,000 magnetic nanoparticles.

14. The nanostructure of claim 11, wherein the nanostructure has a diameter of from about 100 nm to about 1,000 nm.

15. The nanostructure of claim 11, wherein the target membrane protein comprises a transmembrane protein.

16. The nanostructure of claim 11, wherein the target membrane protein comprises TrkB or FZD1.

17. The nanostructure of claim 11, wherein the cell membrane-derived material is obtained from a cell membrane of a human cell.

18. The nanostructure of claim 17, wherein the human cell is a neuronal cell.

19. The nanostructure of claim 11, wherein the magnetic nanoparticles comprise a surface coating that is negatively charged.

20. The nanostructure of claim 19, wherein the surface coating comprises a moiety selected from tannic acid, a gluconic acid, a citric acid, a glutathione, a quinic acid, a lactobionic acid, a dopamine, a polyacrylic acid, or a combination thereof.

21. The nanostructure of claim 11, wherein the nanostructure further comprises a binding agent that selectively binds to the target membrane protein.

22. The nanostructure of claim 21, wherein the binding agent is a phytochemical.

23. The nanostructure of claim 21, wherein the binding agent comprises a pharmacological agent useful for treating a disease.
